(19) United States
(12) Patent Application Publication
(10) Pub. No.: US 2018/0344881 A1
(43) Pub. Date: Dec. 6, 2018

(54) FORMULATIONS FOR 2-HETEROARYL SUBSTITUTED BENZOFURANS

(71) Applicant: Navidea Biopharmaceuticals, Inc., Dublin, OH (US)

(72) Inventors: Richard R. Cesati, III, Pepperell, MA (US); David S. Casebier, Carlisle, MA (US); Richard Christian Moreton, Waltham, MA (US)

(21) Appl. No.: 15/777,640

(22) PCT Filed: Nov. 21, 2016
(86) PCT No.: PCT/US16/63154

§ 371 Date: May 18, 2018

Related U.S. Application Data
(60) Provisional application No. 62/258,309, filed on Nov. 20, 2015.
(60) Provisional application No. 62/258,326, filed on Nov. 20, 2015.

(51) Int. Cl. A61K 51/04; A61K 51/12

(52) U.S. Cl. CPC A61K 51/0455; A61K 51/121

(57) ABSTRACT

Processes and compositions for 2-heteroaryl substituted benzofuran derivatives are described. The 2-heteroaryl substituted benzofuran derivatives may be suitable for preparing radiolabeled 2-heteroaryl substituted benzofuran derivatives for imaging amyloid deposits in living patients.

### FIELD OF THE INVENTION

The present invention relates to novel formulations of 2-heteroaryl substituted benzofuran derivatives and their uses, along with processes for creation of such formulations and derivatives.

### BACKGROUND OF THE INVENTION

Amyloidosis is a progressive, incurable metabolic disease of unknown cause characterized by abnormal deposits of protein in one or more organs or body systems. Amyloid proteins are manufactured, for example, by malfunctioning bone marrow. Amyloidosis, which occurs when accumulated amyloid deposits impair normal body function, can cause organ failure or death. It affects males and females equally and usually develops after the age of 40. At least 15 types of amyloidosis have been identified. Each one is associated with deposits of a different kind of protein.

The major forms of amyloidosis are primary systemic secondary, and familial or hereditary amyloidosis. There is also another form of amyloidosis associated with Alzheimer's disease. Primary systemic amyloidosis usually develops between the ages of 50 and 60. With about 2,000 new cases diagnosed annually, primary systemic amyloidosis is the most common form of this disease in the United States. Also known as light-chain-related amyloidosis, it may also occur in association with multiple myeloma (bone marrow cancer). Secondary amyloidosis is a result of chronic infection or inflammatory disease. It is often associated with Familial Mediterranean fever (a bacterial infection characterized by chills, weakness, headache, and recurring fever), Granulomatous ileitis (inflammation of the small intestine), Hodgkin's disease, Leprosy, Osteomyelitis and Rheumatoid arthritis.

Familial or hereditary amyloidosis is the only inherited form of the disease. It occurs in members of most ethnic groups, and each family has a distinctive pattern of symptoms and organ involvement. Hereditary amyloidosis is thought to be autosomal dominant, which means that only one copy of the defective gene is necessary to cause the disease. A child of a parent with familial amyloidosis has a 50-50 risk of developing the disease.

Amyloidosis can involve any organ or system in the body. The heart, kidneys, gastrointestinal system, and nervous system are affected most often. Other common sites of amyloid accumulation include the brain, joints, liver, spleen, pancreas, respiratory system, and skin.

Alzheimer's disease (AD) is the most common form of dementia, a neurologic disease characterized by loss of mental ability severe enough to interfere with normal activities of daily living, lasting at least six months, and not present from birth. AD usually occurs in old age, and is marked by a decline in cognitive functions such as remembering, reasoning, and planning.

Between two and four million Americans have AD; that number is expected to grow to as many as 14 million by the middle of the 21st century as the population as a whole ages. While a small number of people in their 40s and 50s develop the disease, AD predominantly affects the elderly. AD affects about 3% of all people between ages 65 and 74, about 20% of those between 75 and 84, and about 50% of those over 85. Slightly more women than men are affected with AD, even when considering women tend to live longer, and so there is a higher proportion of women in the most affected age groups. The accumulation of amyloid Aβ-peptide in the brain is a pathological hallmark of all forms of AD. It is generally accepted that deposition of cerebral amyloid Aβ-peptide is the primary influence driving AD pathogenesis. (Hardy J and Selkoe D. J., Science, 297: 353-356, 2002).

Imaging techniques, such as positron emission tomography (PET) and single photon emission computed tomography (SPECT), are effective in monitoring the accumulation of amyloid deposits in the brain and correlating it to the progression of AD (Shoghi-Jadid et al. The American journal of Geriatric Psychiatry, 2002, 10, 24; Miller, Science, 2006, 313, 25 1376; Coimbra et al. Curr. Top. Med. Chem., 2006, 6, 629; Nordberg, Lancet Neural., 2004, 3, 519). The application of these techniques requires the development of radioligands that readily enter the brain and selectively bind to amyloid deposits in vivo. A need exists for amyloid binding compounds that can cross the blood-brain barrier, and consequently, can be used in diagnostics. Furthermore, it is important to be able to monitor the efficacy of the treatment given to AD patients, by measuring the effect of said treatment by measuring changes of AD plaque level.

Properties of particular interest of a detectable amyloid-binding compound, besides high affinity for amyloid deposits in vivo and high and rapid brain entrance, include low unspecific binding to normal tissue and rapid clearance from the same. These properties are commonly dependent on the lipophilicity of the compound (Coimbra et al. Curr. Top. Med. Chem. 2006, 6, 629). Among the proposed small molecules for imaging amyloid plaques, some uncharged analogs of thioflavin T of potential use have been synthesized (Mathis et al. J. Med. Chem. 2003, 46, 2740). Different isosteric heterocycles are reported as potential amyloid binding ligands (Cai et al. J. Med. Chem. 2004, 47, 2208; Kung et al. J. Med. Chem. 2003, 46, 237). Benzofuran derivatives have previously been described for use as amyloid imaging agents (Ono et al. J. Med. Chem. 2006, 49, 2725; Lockhart et al. J. Biol. Chem. 2005, 280(9), 7677; Kung et al. Nuclear Med. Biol. 2002, 29(6), 633; WO2003051859 and for use in preventing Abeta aggregation (Twyman et al. Tetrahedron Lett. 1999, 40(52), 9383; Howlett et al. Biochemical Journal 1999, 340(1), 283; Choi et al. Archives of Pharmacal Research 2004, 27(1), 19; Twyman et al. Bioorg. Med. Chem. Lett. 2001, 11(2), 255; WO9517095).

Benzothiophene derivatives have previously been described for use as amyloid imaging agents (Chang et al. Nuclear Medicine and Biology 2006, 33, 811) and for use as neuroprotectant against amyloid toxicity (JP11116476). There is a need for improved compounds to obtain a signal-to-noise ratio high enough to allow detailed detection of amyloid deposits throughout all brain regions, and providing improved reliability in quantitative studies on amyloid plaque load in relation to drug treatments.

### FIGURES

FIG. 1 shows data from retention of [¹⁸F]NAV4694 on Sartorius Minisart 16596 HY 0.2 μm using PEG400.

FIG. 2 shows a synthetic route to tert-butyl [5-(5-(ethoxymethoxy)-2-benzofuranyl)-(6-nitro-2-pyridinyl)](methyl)carbamate.

FIG. 3 shows an example of preparation of 5-Methoxybenzofuran.

FIG. 4 shows an example of a process for demethylation of 5-Methoxybenzofuran.

FIG. 5 shows an example of a process for alkylation of 5-benzofuranol.

FIG. 6 shows an example of a process for bromination of 3-hydroxy-2-nitropyridine.

FIG. 7 shows an example of a Buchwald amidation of 2-bromo-5-hydroxy-6-nitropyridine.

FIG. 8 shows an example of a process for formation of a triflate ester as disclosed herein.

FIG. 9 shows an example of lithiation and boronylation of a benzofuran as disclosed herein and coupling to the pyridine moiety.

### DETAILED DESCRIPTION

Embodiments of the present invention solve many of the problems and/or overcome many of the drawbacks and disadvantages of the prior art by providing systems and methods for 2-heteroaryl substituted benzofuran derivatives.

Certain of the formulations may comprise an imaging agent with formula:

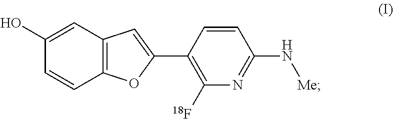 [SMILES: CNc1ccc(-c2cc3cc(O)ccc3o2)c([18F])n1]

and a pharmaceutically acceptable solubilizing excipient comprising polyethylene glycol (PEG), preferably PEG 200-400, more preferably PEG 250-350, and most preferably PEG 300.

Certain embodiments of the formulation comprise less than about 65% polyethylene glycol (PEG), preferably PEG 200-400, more preferably PEG 250-350, and most preferably PEG 300. In some embodiments, the formulation can comprise less than about 75%, less than about 60%, or less than about 50% PEG, PEG 200-400, PEG 250-250, or PEG 300.

Certain embodiments of the formulation comprise between about 0% and about 50%, or between about 1% and about 15%, or between about 5% and about 10% polyethylene glycol (PEG), preferably PEG 200-400, more preferably PEG 250-350, and most preferably PEG 300.

Certain embodiments of the formulation comprise about 8% polyethylene glycol (PEG), preferably PEG 200-400, more preferably PEG 250-350, and most preferably PEG 300.

Certain embodiments of the present invention can comprise ethanol.

Certain embodiments of the formulation may comprise less than about 5% ethanol and less than about 15% polyethylene glycol (PEG), preferably PEG 200-400, more preferably PEG 250-350, and most preferably PEG 300.

Certain embodiments of the formulation may comprise about 3% ethanol and about 8% polyethylene glycol (PEG), preferably PEG 200-400, more preferably PEG 250-350, and most preferably PEG 300.

Certain embodiments of the invention may comprise a stabilizer.

Certain embodiments of the invention may comprise a stabilizer and the stabilizer may comprise ascorbic acid or a salt thereof. In some embodiments, a stabilizer can comprise gentisic acid.

Certain embodiments of the formulation comprise less than about 20 mg/mL ascorbic acid or salt thereof and less than about 15% polyethylene glycol (PEG), preferably PEG 200-400, more preferably PEG 250-350, and most preferably PEG 300.

Certain embodiments of the formulation comprise about 4 mg/mL ascorbic acid or salt thereof and about 8% polyethylene glycol (PEG), preferably PEG 200-400, more preferably PEG 250-350, and most preferably PEG 300.

Certain embodiments of the invention may comprise a buffer.

Certain embodiments may comprise a buffer, the buffer comprising phosphoric acid or a salt thereof. In some embodiments, a buffer can comprise monosodium phosphate, disodium phosphate, monopotassium phosphate, dipotassium phosphate, and combinations thereof.

Certain embodiments of the formulation comprise less than about 5 mg/mL phosphoric acid or salt thereof and less than about 15% polyethylene glycol (PEG), preferably PEG 200-400, more preferably PEG 250-350, and most preferably PEG 300.

Certain embodiments of the formulation comprise about 1 mg/mL phosphoric acid or salt thereof and about 8% polyethylene glycol (PEG), preferably PEG 200-400, more preferably PEG 250-350, and most preferably PEG 300.

Certain embodiments of the formulation comprise a buffer comprising less than about 1 mg/mL sodium phosphate dibasic.

Certain embodiments of the formulation can comprise a buffer and the buffer can be used to control the pH of the formulation.

Certain embodiments of the formulation may have a pH that is between about 5 and about 8.

Certain embodiments of the formulation may have a pH that is about 7.

Certain embodiments may comprise a salt. In some embodiments the salt can comprise sodium chloride.

Certain embodiments of the formulation comprise less than about 10 mg/mL sodium chloride and less than about 15% polyethylene glycol (PEG), preferably PEG 200-400, more preferably PEG 250-350, and most preferably PEG 300.

Certain embodiments of the formulation can comprise about 7 mg/mL sodium chloride and about 8% polyethylene glycol (PEG), preferably PEG 200-400, more preferably PEG 250-350, and most preferably PEG 300.

Certain embodiments of the formulation can comprise a salt and the salt can be used to control the tonicity of the formulation.

Certain embodiments of the invention can be isotonic and the formulation can be isotonic.

Certain embodiments may comprise ethanol and/or a stabilizer.

Certain embodiments of the formulation may comprise a stabilizer and the stabilizer can comprise ascorbic acid or a salt thereof.

Certain embodiments of the formulation can comprise less than about 5% ethanol, less than about 20 mg/mL ascorbic acid or salt thereof, and less than about 15% polyethylene glycol (PEG), preferably PEG 200-400, more preferably PEG 250-350, and most preferably PEG 300.

Certain embodiments of the formulation comprise about 3% ethanol, about 4 mg/mL ascorbic acid or salt thereof, and about 8% polyethylene glycol (PEG), preferably PEG 200-400, more preferably PEG 250-350, and most preferably PEG 300.

Certain embodiments of the invention can comprise a stabilizer and/or a buffer.

Certain embodiments can comprise a stabilizer and the stabilizer can comprise ascorbic acid or a salt thereof and further comprise a buffer and the buffer can comprise phosphoric acid or a salt thereof.

Certain embodiments of the invention can comprise ethanol, a stabilizer, and/or a buffer.

Certain embodiments of the invention can comprise a stabilizer, and the stabilizer can comprise ascorbic acid or a salt thereof, and a buffer comprising phosphoric acid or a salt thereof.

Certain embodiments of the formulation can comprise less than about 20 mg/mL ascorbic acid or salt thereof, less than about 5 mg/mL phosphoric acid or salt thereof, and less than about 15% polyethylene glycol (PEG), preferably PEG 200-400, more preferably PEG 250-350, and most preferably PEG 300.

Certain embodiments of the formulation comprise about 4 mg/mL ascorbic acid or salt thereof, about 1 mg/mL phosphoric acid or salt thereof, and about 8% polyethylene glycol (PEG), preferably PEG 200-400, more preferably PEG 250-350, and most preferably PEG 300.

Certain embodiments of the formulation comprise sodium ascorbate and/or sodium phosphate.

Certain embodiments of the invention can comprise ethanol, a stabilizer, a buffer, and/or a salt.

Certain embodiments of the invention can comprise a stabilizer, the stabilizer comprising ascorbic acid or a salt thereof, a buffer comprising phosphoric acid or a salt thereof, and a salt comprising sodium chloride.

Certain embodiments of the formulation comprise less than about 20 mg/mL ascorbic acid or salt thereof, less than about 5 mg/mL phosphoric acid or salt thereof, less than about 10 mg/mL sodium chloride, and less than about 15% polyethylene glycol (PEG), preferably PEG 200-400, more preferably PEG 250-350, and most preferably PEG 300.

Certain embodiments of the formulation comprise about 4 mg/mL ascorbic acid or salt thereof, about 1 mg/mL phosphoric acid or salt thereof, about 7 mg/mL sodium chloride, and about 8% polyethylene glycol (PEG), preferably PEG 200-400, more preferably PEG 250-350, and most preferably PEG 300.

Certain embodiments of the formulation comprise about 4 mg/mL sodium ascorbate, about 0.8 mg/mL sodium phosphate dibasic, about 7 mg/mL sodium chloride, and about 8% polyethylene glycol (PEG), preferably PEG 200-400, more preferably PEG 250-350, and most preferably PEG 300.

Certain embodiments of the formulation comprise between about 0.5 mCi/mL and about 75 mCi/mL of an imaging agent comprising formula (I).

Certain embodiments of the formulation comprise between about 0.5 mCi/mL and about 65 mCi/mL, or between about 1 mCi/mL and about 50 mCi/mL, or between about 2 mCi/mL and about 25 mCi/mL, or between about 5 mCi/mL and about 15 mCi/mL of an imaging agent comprising formula (I).

Certain embodiments of the formulation can be essentially free of methanol and/or acetonitrile.

Certain embodiments of the formulation comprise less than about 1 μg/mL of the compound comprising formula:

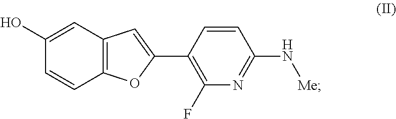 [SMILES: CNc1ccc(-c2cc3cc(O)ccc3o2)c(F)n1]

or a salt thereof.

In certain embodiments, a formulation can comprise a solubilizing excipient used to reduce filter retention of an imaging agent comprising formula (I).

In certain embodiments, a formulation can comprise a solubilizing excipient and the solubilizing excipient can be used to reduce filter retention of an imaging agent comprising formula (I) during filtration.

Certain embodiments can comprise a solubilizing excipient used to reduce filter retention of an imaging agent comprising formula (I) during administration.

Certain embodiments can comprise an imaging agent comprising formula (I) that can be administered using a catheter. In some embodiments, administration can be done intravenously.

Certain embodiments of the invention may have retention during filtration that can be between about 0% and about 15%, or between 1% and about 10%, or between about 1% and about 5% of the total quantity of an imaging agent comprising formula (I).

Certain embodiments of the invention may have retention during administration that can be between about 0% and about 15%, or between 1% and about 10%, or between about 1% and about 5% of the total quantity of an imaging agent comprising formula (I).

Certain embodiments of the invention may have retention during filtration that can be less than about 1%.

Certain embodiments of the invention may have retention during administration that can be less than about 1%.

Certain embodiments of the invention may have radiochemical purity of an imaging agent comprising formula (I) that can be at least about 90%, 91%, 92%, 93%, 94%, 95%, 96%, 97%, 98%, or 99%. In some embodiments, radiochemical purity can be about 90-95%, about 95-98%, at least about 95%, at least about 98%, and ranges therebetween.

Certain embodiments of the invention may have radiochemical purity of an imaging agent comprising formula (I) that can be at least about 90% for at least about 1 hour, 2 hours, 3 hours, 4 hours, 5 hours, 6 hours, 7 hours, 8 hours, 9 hours, 10 hours, 11 hours, or 12 hours.

Certain embodiments of the invention may have radiochemical purity of an imaging agent comprising formula (I) that can be at least about 90% for at least about 10 hours.

Certain embodiments of the invention can comprise a method of synthesizing an imaging agent comprising formula:

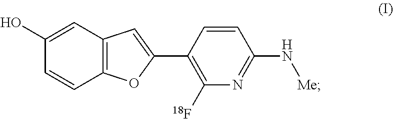 [SMILES: CNc1ccc(-c2cc3cc(O)ccc3o2)c([18F])n1]

the method comprising:
(A) reacting precursor compounds comprising formulae:

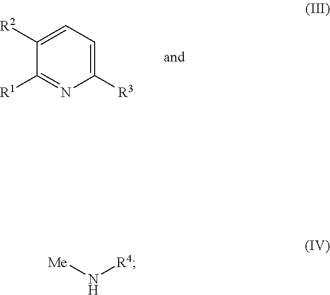

wherein:
R¹ can be a hydrogen or a leaving group;
R² and R³ can be the same or different, and are selected from the group consisting of hydrogen, hydroxyl and halide;
R⁴ can be hydrogen or a nitrogen protecting group;

to produce a compound comprising formula:

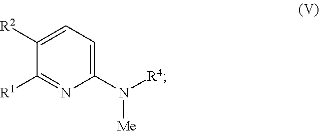

wherein:
R¹ can be hydrogen or a leaving group;
R² can be hydrogen, hydroxyl or halide; and

R⁴ can be hydrogen or a nitrogen protecting group;
(B) reacting the compound comprising formula (V) with a compound comprising formula:

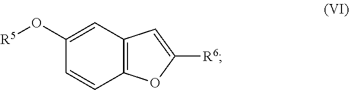

wherein:
   R⁵ can be hydrogen or an oxygen protecting group; and

R⁶ can be hydrogen or halide;

to produce compound comprising formula:

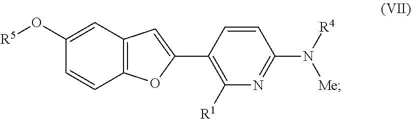

wherein:
R¹ can be hydrogen or a leaving group;

R⁴ can be hydrogen or a nitrogen protecting group; and

R⁵ can be hydrogen or an oxygen protecting group;
(C) reacting the compound comprising formula (VII) with an ¹⁸F species to form an imaging agent precursor comprising formula:

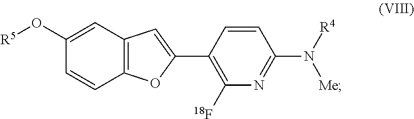

wherein:
R⁴ can be hydrogen or a nitrogen protecting group; and
R⁵ can be hydrogen or an oxygen protecting group;

(D) reacting the imaging agent precursor comprising formula (VIII) under conditions suitable to form the imaging agent comprising formula (I).

In certain embodiments, leaving group of R¹ may be selected from cyano, nitro, halide, trialkylammonium, or aryliodonium.

Certain embodiments may comprise a method for synthesizing a precursor to an imaging agent comprising formula:

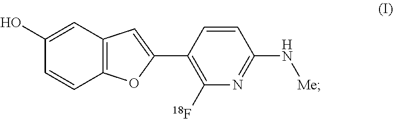 [SMILES: CNc1ccc(-c2cc3cc(O)ccc3o2)c([18F])n1]

the method comprising:

reacting a compound comprising formula:

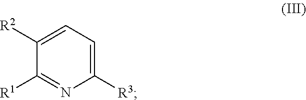

wherein:
R¹ can be hydrogen or a leaving group;
R² and R³ can be the same or different, and are selected from the group consisting of hydrogen, hydroxyl and halide;

with a compound comprising formula:

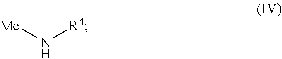

wherein:

R⁴ can be hydrogen or a nitrogen protecting group;
to produce a compound comprising formula:

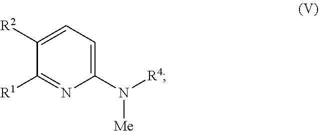

wherein:
R¹ can be hydrogen or a leaving group;
R² can be hydrogen, hydroxyl or halide; and

R⁴ can be hydrogen or a nitrogen protecting group;

wherein the reacting occurs in the presence of a metal catalyst.

In certain embodiments, the metal catalyst may comprise palladium and/or iridium.

In certain embodiments, R¹ may be NO₂; R² may be hydroxyl; R³ may be halide; and R⁴ may be a nitrogen protection group.

In certain embodiments, the compound comprising formula (III) may be the structure:

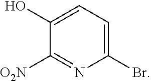 [SMILES: O=[N+]([O-])c1nc(Br)ccc1O]

In certain embodiments, the compound comprising formula (IV) may be the structure:

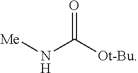

In certain embodiments, the method may comprise reacting a compound comprising formula:

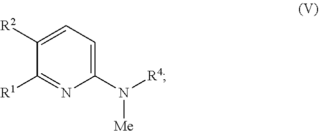

wherein:
R¹ can be hydrogen or a leaving group;
R² can be hydrogen, hydroxyl or halide; and
R⁴ can be hydrogen or a nitrogen protecting group;

with a compound comprising formula:

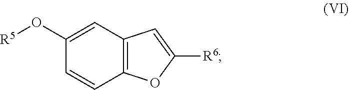

wherein:
R⁵ can be hydrogen or an oxygen protecting group; and
R⁶ can be hydrogen or halide; to produce a compound comprising formula:

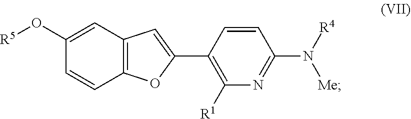

wherein:
R¹ can be hydrogen or a leaving group;
R⁴ can be hydrogen or a nitrogen protecting group; and
R⁵ can be hydrogen or an oxygen protecting group;

wherein the reacting may occur in the presence of a metal catalyst.

In certain embodiments, a metal catalyst may comprise palladium.

In certain embodiments, R¹ may be NO₂; R² may be hydroxyl; R⁴ may be a nitrogen protecting group; R⁵ may be an oxygen protecting group; and R⁶ may be hydrogen or halide.

In certain embodiments, the compound comprising formula (VII) may be the structure:

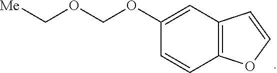 [SMILES: CCOCOc1ccc2occc2c1]

In certain embodiments, the method may comprise reacting the compound comprising formula:

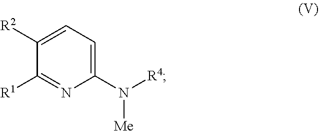

wherein:
R¹ can be hydrogen or a leaving group;
R² can be hydroxyl; and
R⁴ can be hydrogen or a nitrogen protecting group;

with a sulfonyl-containing species to produce a sulfonate-containing compound.

In certain embodiments, the sulfonyl-containing species may be mesyl, tosyl, or trifyl.

In certain embodiments, the sulfonate-containing compound may be the structure:

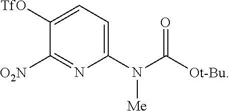 [SMILES: CN(C(=O)OC(C)(C)C)c1ccc(OS(=O)(=O)C(F)(F)F)c([N+](=O)[O-])n1]

In certain embodiments, the method may comprise reacting the compound comprising formula:

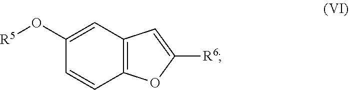

wherein:
R⁵ can be hydrogen or an oxygen protecting group; and
R⁶ can be hydrogen or halide;

with a boron-containing species to produce a boron-containing compound.

In certain embodiments, reacting may occur in the presence of a base.

In certain embodiments, the base may be selected from the group consisting of methyl lithium, n-butyl lithium, sec-butyl lithium, and t-butyl lithium, and combinations thereof.

In certain embodiments, the boron-containing species may be selected from the group consisting of trimethyl borate, triethyl borate, triisopropyl borate, tributyl borate, and tri(2-ethylhexyl) borate, and combinations thereof.

In certain embodiments, the boron-containing compound may be the structure:

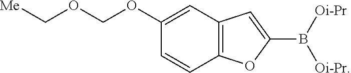

In certain embodiments, the compound comprising formula (VII) may be the structure:

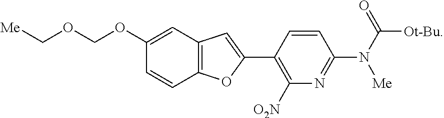 [SMILES: CCOCOc1ccc2oc(-c3ccc(N(C)C(=O)OC(C)(C)C)nc3[N+](=O)[O-])cc2c1]

Some embodiments comprise a method for producing an imaging agent. This imaging agent can be the imaging agent having Formula I. Such a method can comprise the steps of: preparing water for injection (WFI); adding ascorbic acid to the WFI; mixing the WFI and the ascorbic acid; preparing a sodium phosphate dibasic solution by mixing sodium phosphate dibasic with WFI; filtering the sodium phosphate dibasic solution; mixing sodium dibasic solution with the ascorbic acid solution and polysorbate-80; and eluting the imaging agent with ethanol.

In some embodiments, an ascorbic acid solution can be present in a ratio of 2:1 to a sodium dibasic solution. In some embodiments, this ratio can be 1:2, 3:1, 2.5:1 and ranges therebetween.

In some embodiments, a sodium dibasic solution can be present in a ratio of about 4:1 to a polysorbate-80 solution. In some embodiments, this ratio can be about 2:1, about 3:1, about 4.5:1, about 5:1, and ranges therebetween.

Some embodiments comprise a method for producing an imaging agent. This imaging agent can be the imaging agent having Formula I. Such a method can comprise the steps of: preparing water for injection (WFI); adding ascorbic acid to the WFI; mixing the WFI and the ascorbic acid; preparing a sodium phosphate dibasic solution by mixing sodium phosphate dibasic with WFI; filtering the sodium phosphate dibasic solution; preparing a sodium chloride solution; mixing sodium dibasic solution with the ascorbic acid solution, PEG300, the sodium chloride solution; and eluting the imaging agent with ethanol.

In some embodiments, an ascorbic acid solution can be present in a ratio of about 8:1 to sodium phosphate dibasic solution. In some embodiments, this ratio can be about 6:1, about 7:1, about 8.1:1, about 8.5:1, about 9:1, about 10:1, and ranges therebetween. In some embodiments, a sodium chloride solution can be present in a ratio of about 12.25:1 to a sodium phosphate dibasic solution. In some embodiments, this ratio can be about 10:1, about 11:1, about 12:1, about 12.5:1, about 13:1, about 13.5:1, about 14:1, and ranges therebetween.

In some embodiments, PEG300 and equivalents thereof, can be present in a ratio of about 2:1 to a sodium phosphate dibasic solution. In some embodiments, this ratio can be about 1.5:1, about 2.5:1, about 3:1, about 4:1 and ranges therebetween.

In some embodiments, PEG300 can be PEG, PEG 200-400, PEG 250-350, and mixtures thereof.

As used herein, the terms formulation, radiotracer, and imaging agent can be used interchangeably. The meaning of these terms will immediately be understood by the skilled artisan given their context in the claims and the description herein.

Additional features, advantages, and embodiments of the invention are set forth or apparent from consideration of the following detailed description, drawings and claims. Moreover, it is to be understood that both the foregoing summary of the invention and the following detailed description are exemplary and intended to provide further explanation without limiting the scope of the invention as claimed.

Certain embodiments described herein may provide 2-heteroaryl substituted benzofuran derivatives for use as precursors to amyloid imaging agents and treatment of amyloid related diseases, as well as other uses.

Imaging agent 2-[2-(¹⁸F)fluoro-6-(methylamino)-3-pyridinyl)-5-benzofuranol (CAS 1211333-21-9), also known as [¹⁸F]AZ12941236, [¹⁸F]AZD4694, [¹⁸F]NAV4694, AZD4694, and NAV4694, is a short-lived radiotracer labeled with ¹⁸F-fluorine. This material has a short expiry by virtue of the short half-life (109.7 minutes) of ¹⁸F-fluorine. Therefore, a radiotracer, which can also be referred to as an imaging agent herein, such as those disclosed herein can be prepared at distributed facilities using a robust precursor and a dependable radiosynthetic method. The recent requirement by the U.S. Food and Drug Administration (FDA) that these materials must be prepared under the current Good Manufacturing Practices (cGMP) places further importance on the reliability of the method and stability of the compound for production of the desired radiotracer.

U.S. Pat. No. 8,193,363 discloses a formulation of 2-[2-(¹⁸F)fluoro-6-(methylamino)-3-pyridinyl)-5-benzofuranol in a mixture of phosphate buffered saline (pH 7.4) and ethanol (70%) in propylene glycol, 5:3 (v/v), and is herein incorporated by reference in its entirety. The data and rationale for selection of this formulation, however, was insufficient for a commercial product.

Embodiments of formulations as described herein may (1) have an excellent safety profile, (2) allow for efficient passage of the drug product through sterilization filters, and (3) not interfere with the pharmacokinetics of the compound and impart stability. For a radioimaging agent this may include the use of a solubilizing agents and radical scavenging agents such as ascorbic acid, sodium ascorbate, gentisic acid, similar antioxidants, or combinations thereof. Solubilizing agents may comprise: glycerol, polyoxy 15 hydroxy stearate and polyoxy castor oil (Kolliphors), cyclodextrins (α,β, γ), hydroxypropyl betadex, polyvinylpyrrolidinone (povidones), propylene glycol (PG), polyethylene glycol (PEG) (of various molecular weights, 200-400), vitamin E, PEG succinate, and poloxamer 188, and combinations thereof.

The preparation of 2-[2-(¹⁸F)fluoro-6-(methylamino)-3-pyridinyl)-5-benzofuranol may be performed by SNAr2 displacement of a nitro (—NO₂) group from a pyridine ring. Some functional groups may be incompatible and may be destroyed or displaced by general direct fluorination conditions. As such, protective groups may be used to maintain the integrity of the molecule. Additionally, some groups present in a target molecule may interfere with the reaction and may hinder efficient rapid fluorination. These protective groups may be stable to fluorination conditions and be efficiently removed subsequent to the fluorination reaction. Since the majority of nucleophilic fluorinations are performed under basic conditions, groups that are stable to base and are removed by acidic conditions may be preferable. Thus, the phenolic hydroxyl on the benzofuran portion may be protected by an ethoxymethyl ether, and the methylamine on the pyridine portion may be protected as a tert-butyl carbamate.

A precursor for preparation of 2-[2-(¹⁸F)fluoro-6-(methylamino)-3-pyridinyl)-5-benzofuranol may be tert-butyl [5-(5-(ethoxymethoxy)-2-benzofuranyl)-(6-nitro-2-pyridinyl)](methyl)carbamate (CAS 1211333-20-8), also known as AZ13040214 and NAV4614, and is described in U.S. Pat. No. 8,193,363, which is herein incorporated by reference in its entirety.

Certain embodiments may provide processes for preparation of tert-butyl [5-(5-(ethoxymethoxy)-2-benzofuranyl)-(6-nitro-2-pyridinyl)](methyl)carbamate and related intermediates.

Certain embodiments of the invention can comprise a process for preparing Compound 4′. Compound 4′ may be a boron-containing compound. In certain embodiments, the boron-containing compound may be the following:

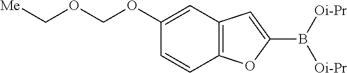

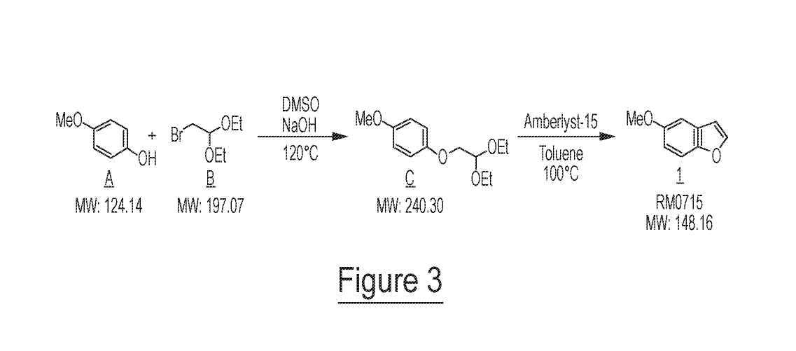

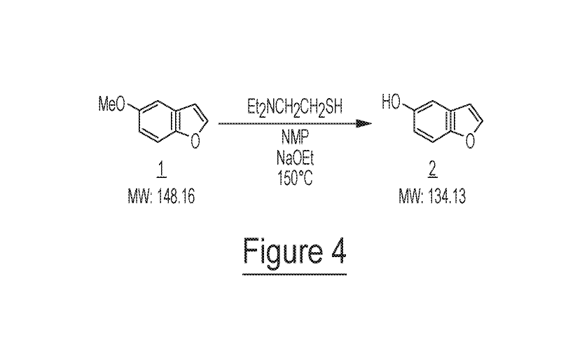

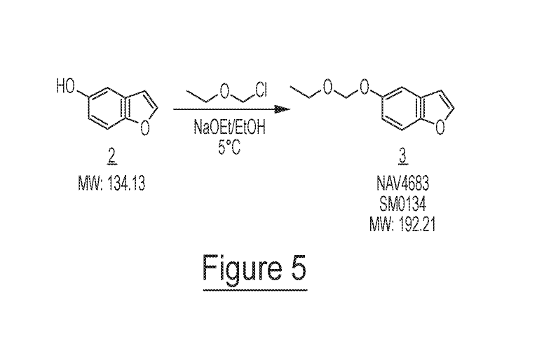

A process for preparing Compound 4′ may include one or more of the following steps:
converting compound A as shown in FIG. 3 to compound C as shown in FIG. 3 in the presence of a base in a solvent;
converting compound C to 5-methoxybenzofuran in the presence of an acidic catalyst as shown in FIG. 3;
converting 5-methoxy benzofuran to 5-benzofuranol in the presence of a dealkylating agent as shown in FIG. 4;
converting 5-benzofuranol into Compound 4′ by a process comprising reacting 5-benzofuranol with a protecting agent in the presence of a base as shown in FIG. 5, and further reacting the protected material with additional base and triisopropyl borate.

In certain embodiments, a base and/or an additional base may be selected from the group of methyl lithium, n-butyl lithium, sec-butyl lithium, t-butyl lithium, and combinations thereof.

The triisopropyl borate may alternatively be selected from the group of trimethyl borate, triethyl borate, triisopropyl borate, tributyl borate, tri(2-ethylhexyl) borate, and combinations thereof.

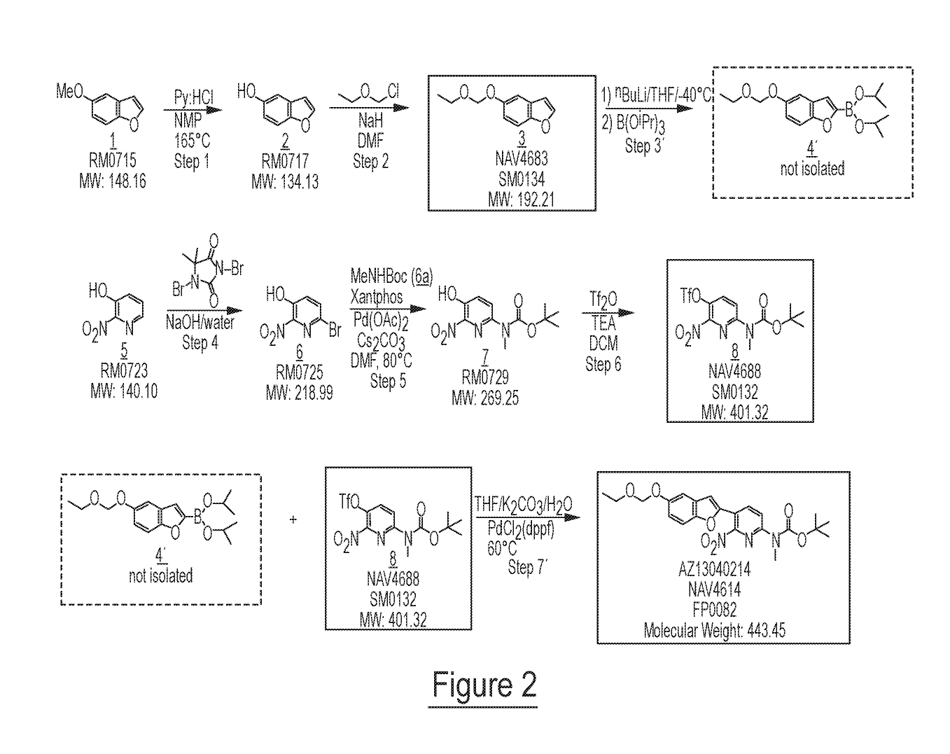

In some embodiments, tert-butyl [5-(5-(ethoxymethoxy)-2-benzofuranyl)-(6-nitro-2-pyridinyl)](methyl)carbamate may be prepared as shown in FIG. 2.

FIG. 2 illustrates a process for production of an end product tert-butyl [5-hydroxy-6-nitro-2-pyridinyl](methyl)carbamate. In certain embodiments, a process for preparation of tert-butyl [5-hydroxy-6-nitro-2-pyridinyl](methyl)carbamate may comprise reaction of tert-butyl methylcarbamate with 2-bromo-5-hydroxy-6-nitropyridine in the presence of a metal catalyst.

In certain embodiments, the metal catalyst may comprise palladium and/or iridium.

### Examples of Preparation of Intermediates

FIG. 3 shows a process for preparation of Compound 1, which is shown in FIG. 2.

An alkylation step may be carried out in dimethyl sulfoxide (DMSO) at approximately 120° C. using approximately 1 equivalent of approximately 50 wt % sodium hydroxide. In certain embodiments the temperature may range from approximately 50° C. to approximately 200° C., more preferably approximately 80° C. to approximately 160° C., more preferably approximately 100° C. to approximately 140° C., and more preferably approximately 110° C. to approximately 130° C. In certain embodiments, approximately 0.5 to approximately 1.5 equivalents of sodium hydroxide may be used. In certain embodiments, sodium hydroxide can be approximately 25 wt % to approximately 75 wt % sodium hydroxide.

The reaction may be quenched with water. The product may be extracted into n-heptane or similar extracting materials. In certain embodiments, unreacted Compound A as shown in FIG. 3 may remain in an aqueous layer. This new process may replace dimethylformamide (DMF) with DMSO and may lower the reaction temperature by approximately 30° C. The product may be isolated in high yields and purity without further purification.

The methyl ether may be cleaved by treatment with 2-diethylaminoethanethiol or similar compounds at approximately 150° C. in N-methylpyrrolidinone or similar compounds under basic (sodium ethoxide or other similar compounds) conditions. In certain embodiments, the temperature may be approximately 100° C. to approximately 200° C. The current conditions may provide cleaner material in an unexpectedly superior yield than those conditions previously reported (such as, for example, approximately 91% yield with greater than approximately 97% purity).

A cyclization step may be carried out in toluene or similar solvents at approximately 100° C. with Amberlyst-15 acidic resin or similar compounds. In certain embodiments, temperature may be approximately 50° C. to approximately 150° C.

Ethanol may be distilled off during the reaction (under Dean Stark conditions, for example) to drive the reaction to completion. Work-up may comprise removal of the resin by filtration, concentration of the solution (Rotavap) and purification of the residue via silica gel chromatography (SiliaFlash G-60, EtOAc/n-heptane).

FIG. 4 illustrates a process for production of Compound 2 from Compound 1 as compared to the process found in Step 1 shown in FIG. 2.

A methyl ether cleavage may be carried out in N-Methyl-2-pyrrolidone (NMP) or similar compounds at approximately 150° C. using approximately 1.2 equivalents of diethylaminoethane thiol and sodium ethoxide or similar compounds. In certain embodiments, the temperature may be approximately 100° C. to approximately 200° C. In certain embodiments, the equivalents may be approximately 0.8 to approximately 1.6.

The mixture may be maintained under a nitrogen atmosphere (to minimize disulfide formation). Ethanol may be distilled off during the process. An aqueous workup may comprise pH adjustment (such as with 1N HCl) then ethyl acetate extractions to isolate crude 5-benzofuranol. The material may then be purified via silica gel chromatography (SiliaFlash G-60, EtOAc/n-heptane).

In an exemplary experiment, a 61.6 g run was carried out which yielded 42.7 g (77%) of the 5-benzofuranol in two batches (98.9% and 99.5% high performance liquid chromatography (“HPLC” or” LC”) purity, respectively). Multiple crops of material may be required to remove non-polar impurities that do not separate during the initial chromatography; the product can be initially isolated in about 94% yield (52.4 g) and about 94.8% purity. Non-polar impurities can cause a reduced quality that can be removed via n-heptane slurry and filtrate can be re-chromatographed to improve recovery.

An unexpected and significant improvement in the preparation may be achieved through the use of diethylaminoethane thiol in place of pyridine hydrochloride, such as described in FIG. 2. This may allow a reduction from approximately 6.0 equivalents to approximately 1.2 equivalents of reagent. A solvent may also be introduced to eliminate running the reaction as a melt, which may lower the reaction temperature from approximately 165-175° C., or as low as about 150° C. in some embodiments.

FIG. 5 shows a process for production of Compound 3 from Compound 2 as shown in FIG. 5 as compared to the process found in Step 2 of FIG. 2.

An etherification may be carried out in ethyl acetate/ethanol at 5° C. with sodium ethoxide (NaOEt) or a similar compound used as a base. An aqueous work-up may be used and the product may be extracted into ethyl acetate/n-heptane or similar solvents, which are readily ascertainable to the skilled artisan. Unreacted 5-benzofuranol may be removed by washing with 1N sodium hydroxide or similar compounds.

In an experiment, a 41.8 g run was carried out which yielded 58.1 g (97%) of 5-(ethoxy)methoxybenzofuranol with a LC purity (98.4%). Chromatography may not be required.

Processes as described herein may remove both sodium hydride (dispersed in mineral oil) and DMF from conditions in the previously reported method and may eliminate the mineral oil which can obviate chromatography prior to further use.

A pyridine portion of the molecule can be assembled per the following procedures. FIG. 5 shows an aspect of Step 4 of an embodiment as shown in FIG. 2.

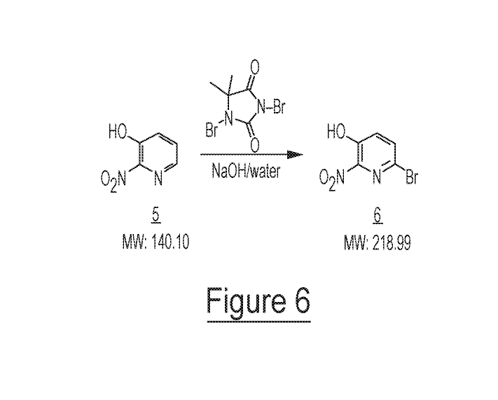

An original process for bromination may utilize 1,3-dibromo-5,5-dimethylhydantoin (DBDMH, Dibromantin) at about 0-5° C. in aqueous sodium hydroxide. In certain embodiments, the temperature may be approximately −5° C. to approximately 10° C. A bromide product as shown as Compound 6 of FIG. 6, may be precipitated directly from the reaction mixture by the addition of acetic acid, and equivalents. Yields by embodiment of such a method can be about 50-55%.

In an experiment, a 176.1 g run was carried out yielding 148.3 g (54%) of 2-bromo-5-hydroxy-6-nitropyridine with an LC purity of 97.8%.

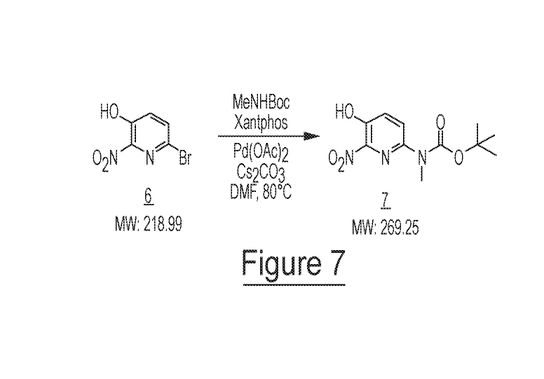

FIG. 7 shows an aspect of Step 5 shown in FIG. 2.

Buchwald amidation may be carried out in DMF or similar compounds at approximately 80° C. using palladium acetate/Xantphos or similar combinations of compounds as the catalyst, and solid cesium carbonate or similar compounds as a base. In certain embodiments, temperatures may be approximately 60° C. to approximately 100° C. Unexpectedly, prior art attempts at this reaction were found to be capricious and would in some instances require multiple days for complete conversion; the phenomenon was exacerbated at large scale. A nitrogen sparge of the reaction mixture during processing may increase the rate of reaction as compared to maintaining a nitrogen headspace. Without being bound by theory, it is believed that a nitrogen sparge overcomes an issue related to the heterogeneous nature of the reaction mixture. Work-up may comprise partitioning with ethyl acetate or similar compounds and aqueous citric acid or similar compounds. This may provide an emulsified mixture that comprises a large volume of precipitated salts. A filtration through Celite or similar materials may remove the solids and break the emulsion. The crude amide shown as Compound 7 in FIG. 7 may be purified via silica gel chromatography using an ethyl acetate/n-heptane or similar solvent system.

A number of solvents were tested for improving the rate of reaction. N-methylpyrrolidone (NMP), 2-methyltetrahydrofuran (MeTHF), and 1,4-dioxane were all tested at small (1 g) scale as potential alternatives for this coupling. NMP offered no improvement in reaction rate. When using MeTHF, the reaction did not proceed to completion. At a small scale, 1,4-dioxane was acceptable, but a 10 g test run gave a slow reaction rate with no improvement over DMF. Tests of the amidation with doubled amounts of catalyst and ligand were initially promising, but the yield of Compound 7 as shown in FIG. 7 suffered greatly due to a large number of unidentified by-products. Replacement of cesium carbonate with potassium carbonate gave a different (uncharacterized) major product, based on liquid chromatography retention time.

Using this procedure, an experiment reacted 132.8 g bromopyridine under the described conditions over 72 hours. The reaction mixture was then diluted with MeTHF and filtered to remove cesium salts. The organic layer was separated to remove a large quantity of impurities and the majority of the residual tert-butyl methylcarbamate. Filtration of the cesium salts also minimized foaming during the subsequent acidification/ethyl acetate extraction step. The crude amidation product was purified via silica gel chromatography (SiliaFlash G-60, n-heptane/EtOAc) yielding 82.8 g (51%) of the tert-butyl [5-hydroxy-6-nitro-2-pyridinyl](methyl)carbamate. The isolated material was 98.7% by LC. Subsequent preparative chromatographic purification was performed; collecting the yellow band that eluted from the column and discarding all other fractions.

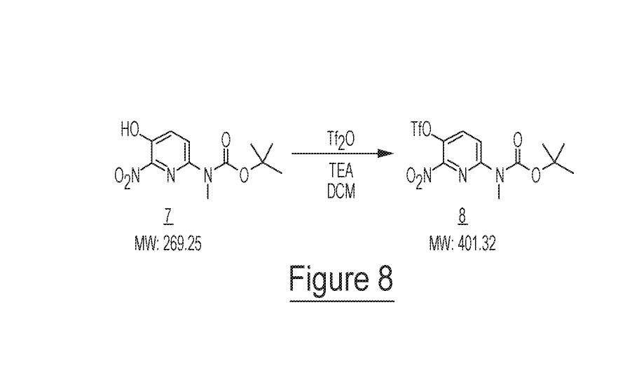

FIG. 8 relates to Step 6 as shown in FIG. 2.

Triflate formation may be carried out using the process shown in FIG. 8. The tert-butyl [5-hydroxy-6-nitro-2-pyridinyl] (methyl) carbamate may be dissolved in DCM or similar compounds and treated with triflic anhydride or similar compounds at approximately −5° C. In certain embodiments, temperatures may be approximately −10° C. to approximately 0° C. The product may then be extracted into MTBE or similar compounds after quenching with water. The crude triflate may be purified via room temperature slurry in 1:1 ethanol-water or similar solution. In an experiment, an 81.7 g run yielded 104.6 g (86%) of tert-butyl [6-nitro-5-trifluoromethylsulfonato-2-pyridinyl] (methyl) carbamate with high purity by LC (99.0%).

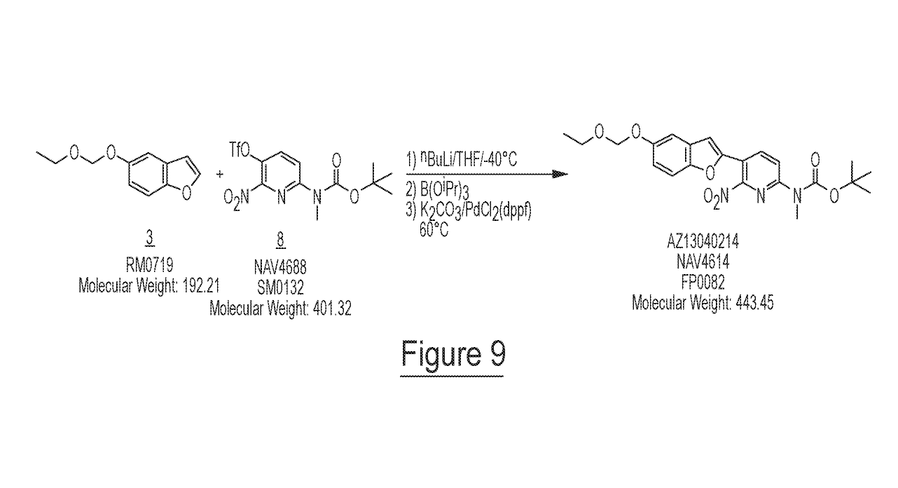

FIG. 9 shows a process for reaction of Compound 3 as shown in FIG. 9, which is also shown in FIG. 2, with Compound 8, which is also shown in FIG. 2. The reaction of Compound 3 with Compound 8 may produce tert-butyl [5-(5-(ethoxymethoxy)-2-benzofuranyl)-(6-nitro-2-pyridinyl)](methyl)carbamate.

A one-pot lithiation/boronylation/Suzuki reaction is shown in FIG. 9, which may produce tert-butyl [5-(5-(ethoxymethoxy)-2-benzofuranyl)-(6-nitro-2-pyridinyl)](methyl)carbamate (NAV4614). The benzofuran may be lithiated at low temperature (approximately −40° C.), or at least −40° C. and converted into a boronic ester via reaction with triisopropyl borate or similar compounds. The resultant boronic ester may then be coupled directly with pyridine triflate or similar compounds at approximately 60° C. using aqueous potassium carbonate or similar compounds as a base and PdCl₂ (dppf) or similar compounds as catalyst. The reaction may be cooled and EtOAc or similar compounds may be added. A silica gel plug preconditioned with 15:85 EtOAc/n-heptane or similar compounds are then used to remove polar impurities and spent catalyst residues. The crude API may be recrystallized from a mixture of MTBE and n-heptane (1:2) or similar compounds. In an experiment, a reaction of 50.2 g of the pyridine under these conditions yielded 65.5 g (57%) of tert-butyl [5-(5-(ethoxymethoxy)-2-benzofuranyl)-(6-nitro-2-pyridinyl)](methyl)carbamate with about 99.3% purity.

### Examples

In examples of experiments, tests were performed on prospective formulations with different filter types to screen for compatibility of the filter material. Both cellulose and polyethersulfone membranes retained unacceptable quantities of the drug substance, with the lowest retention values obtained using polyvinylidene difluoride (PVDF) and polytetrafluoroethylene (PTFE) derived membranes.

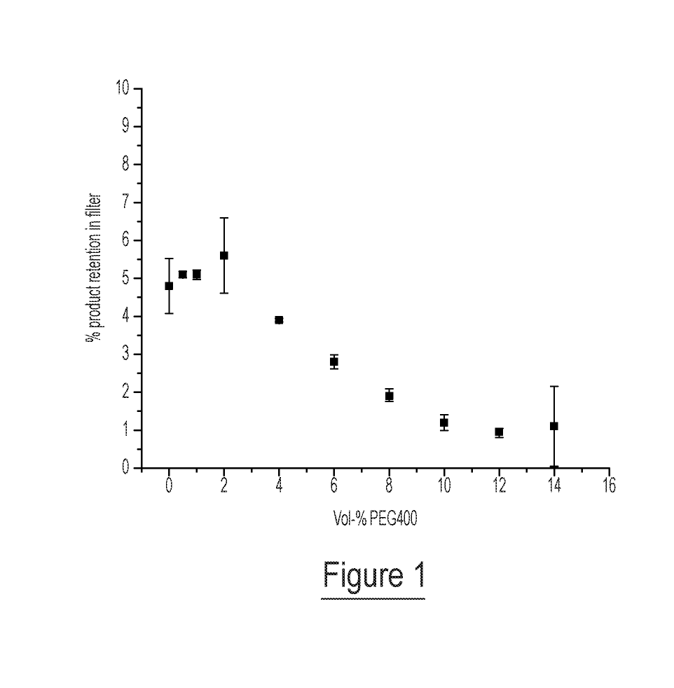

An example of these results is illustrated in FIG. 1, where the retention of the tracer on the filter versus the volume percent of the excipient is plotted. In this case, a solution of [¹⁸F]NAV4694 in PEG400 appears to have low retention at levels of the excipient exceeding 10%.

The filtration times, however, were greater due to increasing solution viscosity with increasing PEG400 concentration. Longer filtration times may reduce the overall yield of the process and may increase the potential risk of filter rupture. The use of Kollidon as an excipient in addition to the PEG400 could reduce this viscosity, but would further complicate the formulation. Detailed evaluation of these latter two filter types was thus performed to test the overall effectiveness of several preferred excipients (Tables 1 and 2).

In the ensuing experiments, each of the excipients were formulated at multiple concentration levels (within acceptable ranges) then treated with small quantities of [¹⁸F]NAV4694 and finally filtered through the preferred filter units. The percent retention of drug substance was then directly calculated through measurement of the actual quantity of radioactivity retained on the filter and compared to that remaining in the filtrate. Results for the Sartorius PVDF (Table 1) and Millipore Millex GV, PVDF (Table 2) filters revealed that both PG and PEG300 displayed improved retention properties over a range of concentration values when compared to alternative excipients.

TABLE 1
Experimental Results for Excipient Screening - Sartorius PVDF

| Excipient | Volume %/24 mL Passage Volume | | | | | |
|---|---|---|---|---|---|---|
| Captisol | | 0 | 0.5 | 1 | 2 | 4 |
| | % retention | 7-9 | 6 | 5-5.5 | 4-4.5 | 3.5 |
| γ-cyclo- | | 0 | 1 | 6 | 12 | |
| dextrin | % retention | 7.5-8.5 | 7-8.5 | 6.5 | 6 | |
| Kolliphor | | 0 | 1 | 5 | 12 | |
| HS15 | % retention | 4.5-9 | 9-9.5 | 9-10 | 6-7 | |
| PG | | 0 | 4 | 8 | 14 | |
| | % retention | 1.5-4 | 1.5 | 1.0 | 0.7 | |
| PEG300 | | 0 | 2 | 4 | 6 | 10 |
| | % retention | 4-8.5 | 5-6 | 4 | 3 | 1 |

TABLE 2
Experimental Results for Excipient Screening - Millex GV

| Excipient | Volume %/24 mL Passage Volume | | | | | |
|---|---|---|---|---|---|---|
| Captisol | | 0 | 0.5 | 1 | 2 | 4 |
| | % retention | 3.5-4 | 0.5-1 | 0.2 | 0.2-2.5 | 2-2.5 |
| γ-cyclo- | | 0 | 1 | 6 | 12 | |
| dextrin | % retention | 1.5-4 | 2.5-4.5 | 2.5-3 | 2.5 | |
| Kolliphor | | 0 | 1 | 6 | 12 | |
| HS15 | % retention | 1.5-4 | 1.5-2.3 | 3-3.5 | 3-3.5 | |
| PG | | 0 | 4 | 8 | 14 | |
| | % retention | 1.5-4 | 1.5 | 0.7-1 | 0.7 | |
| PEG300 | | 0 | 1 | 4 | 8 | 14 |
| | % retention | 1.5-4 | 1.3-1.4 | 0.5-1.75 | 0.5 | 0.3 |

[¹⁸F]NAV4694 formulated with Kolliphor HS15 has higher filter retention than those that are formulated with PG and PEG300.

### 5-methoxybenzofuran (1)

A mixture of 4-methoxyphenol (101.5 g, 0.818 mol), 2-bromo-1,1-diethoxyethane (128.0 mL, 168 g, 0.852 mol) and 50% sodium hydroxide (44.0 mL, 66.7 g, 0.834 mol) in DMSO (1.0 L) was heated at 120° C. for 1.5 hours. The solution was then cooled to 45° C. and transferred into a mixture of 1.0 L of water and 1.0 L of n-heptane. The layers were separated and the organic layer was washed with 250 mL of water and then dried over anhydrous magnesium sulfate. The product solution was then concentrated via rotary evaporation to 166.9 g (85%) of a clear, orange oil.

To a solution of 2-(4-methoxyphenoxy)-1,1-diethoxyethane (166.9 g, 0.695 mol) in toluene (835 mL) was added Amberlyst-15 resin (16.7 g, 10 wt %). The reaction mixture was heated at reflux for a total of 7 h while ethanol was removed via azeotropic distillation with a Dean-Stark trap. The mixture was then cooled to room temperature, filtered and concentrated at the rotary evaporator. The resulting dark oil was twice reconstituted in 200 mL of methanol and concentrated to 96.1 g of residue. The crude product was chromatographed on 1.5 kg of SiliaFlash G-60 using 10% MTBE in n-heptane. 5-methoxybenzofuran was isolated as 40.6 g (39%) of a clear oil.

### 5-Hydroxybenzofuran (2)

A mixture of 5-methoxybenzofuran (61.6 g, 0.416 mol), 2-(diethylamino)ethanethiol: HCl (88.4 g, 0.521 mol) and NMP (600 mL) was sparged with nitrogen for 10 min. Sodium ethoxide solution (388 mL, 21 wt % in EtOH, 1.04 mol) was added and the resulting solution was heated to 150° C. overnight. A Dean-Stark trap was used to distill off ethanol during the reaction. The mixture was cooled to room temperature and acidified with 1.1 L of 1.0N HCl. The solution was then partitioned with 850 mL of ethyl acetate. The layers were separated and the aqueous layer back extracted twice with 300 mL of ethyl acetate. The combined organic extracts were washed twice with 300 mL of water, once with 150 mL of brine and then dried over anhydrous magnesium sulfate and filtered. The filtrate was then concentrated by rotary evaporation to 72.4 g of a dark oil. This residue was purified on SiliaFlash G-60 (2.0 L, ˜1.0 kg) using 10% EtOAc/n-heptane as eluant, providing 52.4 g of material as waxy solids, 94.8% purity by LC with significant non-polar impurities. The material was slurried for one-hour in 200 mL of n-heptane and then filtered. The solids were further washed with 150 mL of n-heptane and air-dried giving 29.7 g of 5-hydroxybenzofuran with an LC purity of 98.9%. The filtrate was concentrated by rotary evaporation to a residue (16.4 g) which was purified on SiliaFlash G-60 (0.90 L, ˜0.45 kg) using a gradient of 10-25% EtOAc in n-heptane as eluant. This provided an additional 13.0 g of with a 99.5% purity, for a total combined yield of 5-hydroxybenzofuran of 42.7 g (77%).

### 5-(Ethoxymethoxy)benzofuran (3)

A solution of 5-hydroxybenzofuran (41.8 g, 0.312 mol) in ethyl acetate (420 mL) was cooled at −7.1° C. Sodium ethoxide solution (21 wt % in EtOH, 131.9 g, 0.407 mol) was added over 5 minutes and then the solution was cooled further to −14.5° C. Chloromethylethyl ether (38.0 mL, 38.7 g, 0.409 mol) was added over 10 minutes (max temp: −5.0° C.) and then the mixture was allowed to warm to room temperature and stirred overnight. Water (250 mL) was added and the two-phase mixture was stirred for 50 minutes. n-Heptane (210 mL) and water (170 mL) were added and then the layers were separated. The organic layer was washed with water (210 mL) and brine (210 mL) and then concentrated on a rotary evaporator to a dark oil. The residue was dissolved in n-heptane (500 mL) and washed with 1.0N sodium hydroxide (200 mL), followed by brine (100 mL). The organic layer was then dried over anhydrous magnesium sulfate, filtered, and the filtrate concentrated to yield 58.1 g of 5-(ethoxymethoxy)benzofuran as an orange oil (97%) with a purity of 98.4% (LC).

### 2-Bromo-5-hydroxy-6-nitropyridine (6)

A solution of 3-hydroxy-2-nitropyridine (176.0 g, 1.256 mol) in aqueous sodium hydroxide (74.0 mL of 50% NaOH in 1.94 L water, 1.40 mol) was cooled at 3.3° C. while dibromantin (198.5 g, 0.694 mol) was added portion-wise over 58 minutes maintaining reaction temperature at or below 4° C. The reaction mixture was then allowed to warm to room temperature overnight. The reaction was quenched with acetic acid (80.0 mL, 1.34 mol), and the resulting slurry was stirred at room temperature for 4 h. The solids were collected by filtration, washed with water (3×300 mL), and then vacuum-dried (35° C.) overnight to give 148.3 g (54%) of 2-bromo-5-hydroxy-6-nitropyridine as a yellow solid with a purity of 97.8% (LC).

### tert-butyl [5-hydroxy-6-nitro-2-pyridinyl](methyl)carbamate (7)

2-Bromo-5-hydroxy-6-nitropyridine (132.8 g, 0.606 mol), cesium carbonate (594.7 g, 1.83 mol), tert-butyl methylcarbamate (116.7 g, 0.890 mol) and anhydrous DMF (4.0 L) were combined and the resultant orange mixture was sparged with nitrogen for 10 minutes. Xantphos (53.1 g, 0.0918 mol) and palladium acetate (13.5 g, 0.0601 mol) were then added and the solution was heated to 80° C. with continuous nitrogen gas sparging for 3 days. The mixture was allowed to cool and stir overnight. 2-Methyltetrahydrofuran (MeTHF, 4.0 L) was added and the resultant slurry was stirred for 2.5 hours and then filtered through Celite. The filter cake was washed with 2×1.0 L of MeTHF. The filtrate was then partitioned with 8.0 L of water. The organic layer was discarded and the aqueous layer acidified to pH 1-2 with 60 mL of 12N HCl. Ethyl acetate (4.0 L) was added and the layers were separated. The organic layer was washed with water (2.0 L). The aqueous layer was back extracted with EtOAc (2×2.0 L). The combined organic extracts were concentrated on a rotary evaporator to a brown oil. The residue was twice dissolved in EtOAc (1.0 L each time) and concentrated again. The brown oil was then dissolved in MTBE (300 mL) and loaded onto a silica gel column (SiliaFlash G-60, 3.0 L, ˜1.5 kg) that was pre-conditioned with 10% EtOAc/n-heptane. The column was then eluted sequentially with 10%, 20% and 30% EtOAc in n-heptane. The fractions were discarded until the eluate was yellow and then collected until cessation of color. Concentration of the combined yellow fractions on a rotary evaporator yields two crops of tert-butyl [5-hydroxy-6-nitro-2-pyridinyl](methyl)carbamate of 61.5 g and 21.3 g, (respective LC purities=98.7% and 97.0%) with a combined yield of 82.8 g (51%).

### tert-butyl [6-nitro-5-trifluoromethylsulfonato-2-pyridinyl] (methyl) carbamate (8)

A solution of tert-butyl [5-hydroxy-6-nitro-2-pyridinyl](methyl)carbamate (81.7 g, 0.303 mol) and triethylamine (84.5 mL, 61.3 g, 0.606 mol) in dichloromethane (800 mL) was cooled at −3.0° C. Triflic anhydride (61.0 mL, 102 g, 0.362 mol) was added over 16 minutes at such a rate that the temperature did not exceed −0.3° C. The mixture was stirred with cooling for 21 minutes and then quenched with water (400 mL). The mixture was allowed to warm to room temperature and then MTBE (1600 mL) was added. The organic layer was separated and washed with 10% citric acid (400 mL), water (400 mL) and brine (200 mL). The volatiles were removed on a rotary evaporator and the residue taken up in 1:1 ethanol: water (800 mL). The slurry was stirred for one hour and then filtered. The solids were washed with 1:1 EtOH: water (200 mL) and then air-dried overnight to yield 104.6 g (86%) of tert-butyl [6-nitro-5-trifluoromethylsulfonato-2-pyridinyl] (methyl) carbamate with a purity of 99.0% (LC).

### tert-butyl [5-(5-(ethoxymethoxy)-2-benzofuranyl)-(6-nitro-2-pyridinyl)](methyl)carbamate

A solution of 5-(ethoxymethoxy)benzofuran (50.2 g, 0.261 mol) in anhydrous THF (500 mL) was cooled at −47° C. while n-Butyllithium (130 mL, 0.325 mol, 2.5M in hexanes) was added over 19 minutes at such a rate that the temperature did not exceed −43° C. The solution was stirred with cooling (less than −40° C.) for 20 minutes and then triisopropyl borate (75.0 mL, 61.1 g, 0.325 mol) was added over 11 minutes (max temp=−43° C.). The mixture was allowed to warm to room temperature over 72 minutes and then a degassed solution of potassium carbonate (108 g, 0.781 mol) in water (700 mL) was added. tert-butyl [6-nitro-5-trifluoromethylsulfonato-2-pyridinyl] (methyl) carbamate (114.4 g, 0.285 mol) and PdCl₂(dppf):DCM (2.27 g, 0.00278 mol) were added and the mixture was degassed and heated to 60° C. for 4 hours. The dark solution was then allowed to cool to room temperature and stirred overnight. Ethyl acetate (800 mL) was added and the layers separated. The organic layer was washed with water (2×250 mL) and brine (200 mL) and then concentrated on a rotary evaporator to a dark oil. The residue was twice dissolved in EtOAc (250 mL) and again concentrated yielding 124.3 g of crude tert-butyl [5-(5-(ethoxymethoxy)-2-benzofuranyl)-(6-nitro-2-pyridinyl)](methyl) carbamate. The material was dissolved in a mixture of ethyl acetate and n-heptane (100 mL each) and then purified via silica gel chromatography on SiliaFlash G-60 (1.0 L, ˜0.5 kg) using n-heptane: EtOAc (85:15) as the eluant. The product containing fractions were combined and concentrated giving 100.2 g of tert-butyl [5-(5-(ethoxymethoxy)-2-benzofuranyl)-(6-nitro-2-pyridinyl)](methyl) carbamate. The material was then dissolved in MTBE (300 mL) at 40° C. and diluted slowly with n-heptane (600 mL) at the same temperature. The resulting solution was cooled to room temperature and seeded with a small amount of product seeds (previously prepared by transferring 1 mL of the supersaturated solution into a small glass vial). The resulting slurry was stirred for 2.5 hours. The solids were filtered and washed with n-heptane (250 mL) and then vacuum dried at room temperature overnight to yield 65.5 g (57%) of tert-butyl [5-(5-(ethoxymethoxy)-2-benzofuranyl)-(6-nitro-2-pyridinyl)](methyl) carbamate with a purity of 99.3% (LC).

One formulation that has been employed for the administration of 2-[2-(¹⁸F)fluoro-6-(methylamino)-3-pyridinyl)-5-benzofuranol uses polysorbate 80 as a solubilizing agent. An example composition is shown below:

### Example: Polysorbate 80 Formulation

| | | |
|---|---|---|
| EtOH | 3.4% | (v/v) |
| NaCl | 0.76% | (w/v) |
| Polysorbate 80 | 0.86% | (v/v) |
| Na₂HPO₄ | 0.83 | mg/mL |
| L-Ascorbate | 4.1 | mg/mL |

This formulation may be generated by elution of the radiotracer (negligible by mass, <1 ug/mL) with 1 mL EtOH into a formulation base made using the following procedure:

a. Prepare WFI with Ascorbic Acid Solution.
Withdraw 1.2±0.1 mL volume from a 10 mL vial of sterile water for injection (WFI).
Add 1.2±0.1 mL of 500 mg/mL of ascorbic acid solution USP to the 8.8 mL of WFI.
Mix the solution.

b. Prepare Sodium Phosphate Dibasic Solution.
Accurately weigh 1000±10 mg of Na₂HPO₄
Withdraw 4.0 mL volume from a 50 mL vial of WFI.
Dissolve the salt in the withdrawn WFI.
Filter the resulting solution through a 0.2 μm sterile filter into the original 50 mL vial.

c. Preparation of Formulation Basis.
Mix 1.2±0.1 mL of sodium phosphate dibasic solution with
2.0±0.1 mL of WFI with ascorbic acid solution and
0.25±0.02 mL of Polysorbate-80 and
24.5±0.2 mL of saline (0.9%) in a freshly opened 30 mL sterile empty vial.
Mix contents well.
(Post synthesis, 1 mL EtOH is added as eluent for the radiotracer)

The drug product solution may be prepared by elution of the radiotracer from the concentration cartridge with ethanol (±1.0 mL).

Polysorbate 80 has been implicated in anaphylactoid reactions in other types of products. Therefore, alternate formulations are desirable to develop and use.

### Example: PEG300 Formulation

| | | |
|---|---|---|
| EtOH | 3.4% | (v/v) |
| NaCl | 0.70% | (w/v) |
| PEG 300 | 8.3% | (v/v) |
| Na₂HPO₄ | 0.83 | mg/mL |
| L-Ascorbate | 4.1 | mg/mL |

This may be generated by elution of the radiotracer (negligible amount by mass, <1 ug/mL) with 1 ml EtOH into a formulation base made). The following procedure outlines an exemplary preparation of the formulation, including the alcohol added as the elution solvent.

a. Prepare WFI with Ascorbic Acid Solution.
Withdraw 1.2±0.1 mL volume from a 10 mL vial of sterile water for injection (WFI).
Add 1.2±0.1 mL of 500 mg/mL of ascorbic acid solution USP to the 8.8 mL of WFI.
Mix the solution.

b. Prepare Sodium Phosphate Dibasic Solution.
Accurately weigh 1000±10 mg of Na₂HPO₄
Withdraw 4.0 mL volume from a 50 mL vial of WFI.
Dissolve the salt in the withdrawn WFI.
Filter the resulting solution through a 0.2 μm sterile filter into the original 50 mL vial.

c. Prepare Formulation Basis
Mix 1.2±0.1 mL of Na₂HPO₄ solution with
2.0±0.1 mL of WFI with ascorbic acid solution and
2.4±0.2 mL of PEG300 and 22.4±0.2 mL of 0.9% (normal) sodium chloride solution in a freshly opened 30 mL sterile empty vial.
Add 1 mL EtOH.
Mix contents well.

The drug product solution may be prepared by elution of the radiotracer from the concentration cartridge with ethanol (±1.0 mL).

### Example: Stability of the Formulation

[¹⁸F]NAV4694 solution for injection was tested over a range of radioactivity concentration values to determine the capacity and potential expiry at given concentrations. At very high activity levels (>100 mCi/mL), lower initial radiochemical purity and accelerated decomposition was observed. At radioactivity concentration values below about 75 mCi/mL, high initial radiochemical purity was observed, and retained over at least ten hours. These results were confirmed in four successive runs, the raw data of which is provided in Table 3.

TABLE 3
[¹⁸F]NAV4694 Stability at Room Temperature, Ambient Humidity

| | Batch Number | | | |
|---|---|---|---|---|
| | X- | X- | X- | X- |
| | 20130828-1 | 20130903-1 | 20130904-1 | 20130910-1* |
| | Activity Conc at EOS | | | |
| Time post | 66 mCi/ml | 64 mCi/ml | 65 mCi/ml | 64 mCi/ml |
| EOS (h) | Radiochemical Purity (%) | | | |
| 0 | 98.0 | 97.8 | 98.8 | 97.9 |
| 0.5 | 97.6 | 97.1 | 97.5 | 98.1 |
| 1 | 97.5 | 95.4 | 95.5 | 96.8 |
| 1.5 | 97.4 | 96.3 | 96.7 | 97.0 |
| 2 | 96.5 | 96.1 | 95.3 | 97.2 |
| 3 | 94.9 | 94.5 | 96.3 | 97.0 |
| 4 | 94.5 | 93.8 | 95.6 | 96.6 |
| 6 | 94.6 | 93.3 | 94.2 | 95.6 |
| 8 | 92.4 | 92.0 | 93.5 | 94.6 |
| 10 | 91.9 | 92.6 | 92.6 | 94.1 |
| 12 | NT | 91.3 | 92.2 | 94.3 |
| 13 | NT | 92.4 | 93.0 | 94.7 |

*batch X-20130910-1 was prepared with seven-day-old phosphate buffer solution;
NT = not tested

Although the foregoing description is directed to the preferred embodiments of the invention, it is noted that other variations and modifications will be apparent to those skilled in the art, and may be made without departing from the spirit or scope of the invention. Moreover, features described in connection with one embodiment of the invention may be used in conjunction with other embodiments, even if not explicitly stated above.

What is claimed:

1. A formulation comprising:
an imaging agent comprising formula: 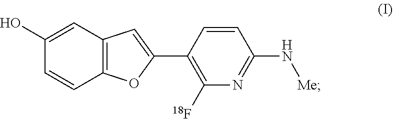 [SMILES: CNc1ccc(-c2cc3cc(O)ccc3o2)c([18F])n1]
and
a pharmaceutically acceptable solubilizing excipient comprising polyethylene glycol 300.

2. The formulation of claim 1, wherein the formulation comprises less than about 65% polyethylene glycol 300.

3. The formulation of claim 2, wherein the formulation comprises between about 0% and about 50%, or between about 1% and about 15%, or between about 5% and about 10% polyethylene glycol 300.

4. The formulation of claim 3, wherein the formulation comprises about 8% polyethylene glycol 300.

5. The formulation of claim 1, further comprising ethanol.

6. The formulation of claim 5, wherein the formulation comprises less than about 15% polyethylene glycol 300 and less than about 5% ethanol.

7. The formulation of claim 5, wherein the formulation comprises about 8% polyethylene glycol 300 and about 3% ethanol.

8. The formulation of claim 1, further comprising a stabilizer.

9. The formulation of claim 8, wherein the stabilizer comprises ascorbic acid or a salt thereof.

10. The formulation of claim 9, where the formulation comprises less than about 15% polyethylene glycol 300 and less than about 20 mg/mL ascorbic acid or salt thereof.

11. The formulation of claim 9, where the formulation comprises about 8% polyethylene glycol 300 and about 4 mg/mL ascorbic acid or salt thereof.

12. The formulation of claim 1, further comprising a buffer.

13. The formulation of claim 12, wherein the buffer comprises phosphoric acid or a salt thereof.

14. The formulation of claim 13, wherein the formulation comprises less than about 15% polyethylene glycol 300 and less than about 5 mg/mL phosphoric acid or salt thereof.

15. The formulation of claim 13, wherein the formulation comprises about 8% polyethylene glycol 300 and about 1 mg/mL phosphoric acid or salt thereof.

16. The formulation of claim 12, wherein the formulation comprises a buffer comprising less than about 1 mg/mL sodium phosphate dibasic.

17. The formulation of claim 12, wherein the buffer controls the pH of the formulation.

18. The formulation of claim 17, wherein the pH of the formulation is between about 5 and about 8.

19. The formulation of claim 17, wherein the pH of the formulation is about 7.

20. The formulation of claim 1, further comprising a salt.

21. The formulation of claim 20, wherein the salt comprises sodium chloride.

22. The formulation of claim 21, wherein the formulation comprises less than about 15% polyethylene glycol 300 and less than about 10 mg/mL sodium chloride.

23. The formulation of claim 21, wherein the formulation comprises about 8% polyethylene glycol 300 and about 7 mg/mL sodium chloride.

24. The formulation of claim 20, wherein the salt controls the tonicity of the formulation.

25. The formulation of claim 24, wherein the formulation is isotonic.

26. The formulation of claim 1, further comprising ethanol and a stabilizer.

27. The formulation of claim 26, wherein the stabilizer comprises ascorbic acid or a salt thereof.

28. The formulation of any one of claims 1 and 27, wherein the formulation comprises less than about 15% polyethylene glycol 300, less than about 5% ethanol, and less than about 20 mg/mL ascorbic acid or salt thereof.

29. The formulation of any one of claims 1 and 27, wherein the formulation comprises about 8% polyethylene glycol 300, about 3% ethanol, and about 4 mg/mL ascorbic acid or salt thereof.

30. The formulation of claim 1, further comprising a stabilizer and a buffer.

31. The formulation of claim 30, wherein the stabilizer comprises ascorbic acid or a salt thereof and the buffer comprises phosphoric acid or a salt thereof.

32. The formulation of any one of claims 1 and 31, wherein the formulation comprises less than about 15% polyethylene glycol 300, less than about 20 mg/mL ascorbic acid or salt thereof, and less than about 5 mg/mL phosphoric acid or salt thereof.

33. The formulation of any one of claims 1 and 31, wherein the formulation comprises about 8% polyethylene glycol 300, about 4 mg/mL ascorbic acid or salt thereof, and about 1 mg/mL phosphoric acid or salt thereof.

34. The formulation of any one of claims 1 and 31, wherein the formulation comprises sodium ascorbate and sodium phosphate.

35. The formulation of claim 1 further comprising ethanol, a stabilizer, and a buffer.

36. The formulation of claim 35, wherein the stabilizer comprises ascorbic acid or a salt thereof, and wherein the buffer comprises phosphoric acid or a salt thereof.

37. The formulation of any one of claims 1 and 35, wherein the formulation comprises less than about 15% polyethylene glycol 300, less than about 20 mg/mL ascorbic acid or salt thereof, and less than about 5 mg/mL phosphoric acid or salt thereof.

38. The formulation of any one of claims 1 and 35, wherein the formulation comprises about 8% polyethylene glycol 300, about 4 mg/mL ascorbic acid or salt thereof, and about 1 mg/mL phosphoric acid or salt thereof.

39. The formulation of any one of claims 1 and 38, wherein the formulation comprises sodium ascorbate and sodium phosphate.

40. The formulation of claim 1, further comprising ethanol, a stabilizer, a buffer, and a salt.

41. The formulation of claim 40, wherein the stabilizer comprises ascorbic acid or a salt thereof, the buffer comprises phosphoric acid or a salt thereof, and the salt comprises sodium chloride.

42. The formulation of claim 41, wherein the formulation comprises less than about 15% polyethylene glycol 300, less than about 20 mg/mL ascorbic acid or salt thereof, less than about 5 mg/mL phosphoric acid or salt thereof, and less than about 10 mg/mL sodium chloride.

43. The formulation of claim 41, wherein the formulation comprises about 8% polyethylene glycol 300, about 4 mg/mL ascorbic acid or salt thereof, about 1 mg/mL phosphoric acid or salt thereof, and about 7 mg/mL sodium chloride.

44. The formulation of claim 41, wherein the formulation comprises about 8% polyethylene glycol 300, about 4 mg/mL sodium ascorbate, about 0.8 mg/mL sodium phosphate dibasic, and about 7 mg/mL sodium chloride.

45. The formulation of claim 41, wherein the pH of the formulation is between about 5 and about 8.

46. The formulation of claim 40, wherein the pH of the formulation is about 7.

47. The formulation of any one of claims 1, 35 and 40, wherein the formulation comprises between about 0.5 mCi/mL and about 75 mCi/mL of the imaging agent comprising formula (I).

48. The formulation of any one of claims 1, 35 and 40, wherein the formulation comprises between about between about 0.5 mCi/mL and about 65 mCi/mL, or between about 1 mCi/mL and about 50 mCi/mL, or between about 2 mCi/mL and about 25 mCi/mL, or between about 5 mCi/mL and about 15 mCi/mL of the imaging agent comprising formula (I).

49. The formulation of any one of claims 1, 38 and 43, wherein the formulation is essentially free of methanol and acetonitrile.

50. The formulation of any one of claims 1, 38 and 43, wherein the formulation comprises less than about 1 μg/mL of the compound comprising formula: 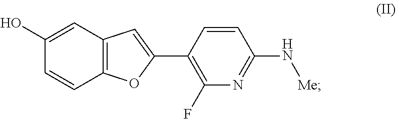 [SMILES: CNc1ccc(-c2cc3cc(O)ccc3o2)c(F)n1]
or a salt thereof.

51. The formulation of claim 1, wherein the solubilizing excipient reduces filter retention of the imaging agent comprising formula (I).

52. The formulation of claim 51, wherein the solubilizing excipient reduces filter retention of the imaging agent comprising formula (I) during filtration.

53. The formulation of claim 51, wherein the solubilizing excipient reduces filter retention of the imaging agent comprising formula (I) during administration.

54. The formulation of claim 53, wherein the imaging agent comprising formula (I) is administered using a catheter.

55. The formulation of claim 52, wherein the retention during filtration is between about 0% and about 15%, or between 1% and about 10%, or between about 1% and about 5% of the total quantity of the imaging agent comprising formula (I).

56. The formulation of claim 53, wherein the retention during administration is between about 0% and about 15%, or between 1% and about 10%, or between about 1% and about 5% of the total quantity of the imaging agent comprising formula (I).

57. The formulation of claim 52, wherein the retention during filtration is less than about 1%.

58. The formulation of claim 53, wherein the retention during administration is less than about 1%.

59. The formulation of any one of claims 1, 35 and 40, wherein the radiochemical purity of the imaging agent comprising formula (I) is at least about 90%, 91%, 92%, 93%, 94%, 95%, 96%, 97%, 98%, or 99%.

60. The formulation of any one of claims 1, 35 and 40, wherein the radiochemical purity of the imaging agent comprising formula (I) is at least about 90% for at least about 1 hour, 2 hours, 3 hours, 4 hours, 5 hours, 6 hours, 7 hours, 8 hours, 9 hours, 10 hours, 11 hours, or 12 hours.

61. The formulation of claim 60, wherein the radiochemical purity of the imaging agent comprising formula (I) is at least about 90% for at least about 8 hours.
